(19) United States
(12) Patent Application Publication
(10) Pub. No.: US 2004/0073988 A1
(43) Pub. Date: Apr. 22, 2004

(54) BUCKLE STRUCTURE OF SWIMMING MASK

(75) Inventor: Pan Chen-Lieh, Ilan Hsing (TW)

Correspondence Address: BRUCE H. TROXELL, 5205 LEESBURG PIKE, SUITE 1404, FALLS CHURCH, VA, 22041, US

(73) Assignee: QDS Injection Molding LLC.

(21) Appl. No.: 10/274,136

(22) Filed: Oct. 21, 2002

(51) Int. Cl. A61F 9/02

(52) U.S. Cl. 2/428

(57) ABSTRACT

An improvement of buckle of swimming mask, said swimming mask comprising a soft mask body, at least a lens, buckle and strap; said mask body and lens are combined integral; the buckle are provided to the mask body in larger area which enable the mask body not easy to misshape which the buckle bearing the strength.

### BACKGROUND OF THE INVENTION

The present invention is relating to a buckle structure of mask which for but not limited to swimming, scuba as well as diving. Said structure is provided between the soft mask part and strap, more particular, especially to the buckle structure of frameless swimming mask.

At present, conventional and known swimming masks normally provide a soft mask, a set of frame and at least one or more lens. The buckle is jointed or mounted to both sides of frame directly. Obviously, the structure is complicated.

Currently, the swimming masks are adopting the frameless structure, that means, the soft mask part are connected with lens directly and the buckle is provided on the soft mask part near the outside of lens. The advantage of this mask is that it can provide wider view and without obstruction. As refer to FIG. 1, in order to prevent the misshape of soft mask B which caused by the unequal pulling strength during adjusting or operating buckle A, therefore, there is a insert-through pin C to increase the surface which enable to share the pulling strength which soft mask B bore. Furthermore, around the insert-through pin C, provide gluey portion D with certain thickness which extending from the soft mask B. By the way, although misshape can be reduced but the viewing obstruction raised by the insert-through pin C and gluey portion D.

### SUMMARY OF THE INVENTION

Therefore, after carefully study, the inventor maturing the present invention. The buckle structure of the present invention is adopting a hard buckle which providing plurality of pins inserting into the holes equally pre-adopting on the soft mask. By the way, the pins are located equally on the larger surface of soft mask part which enable the pulling strength caused by operating and adjusting can be spread in different directions thus decrease the misshape of mask. Therefore, there is no insert-through pin protruded in the inner portion of the mask part.

### BRIEF DESCRIPTION OF THE DRAWINGS

FIG. 1 is the prospective view of the conventional swimming mask.

FIG. 2 is the prospective view of the present invention.

FIG. 3 is the exploit view of the present invention.

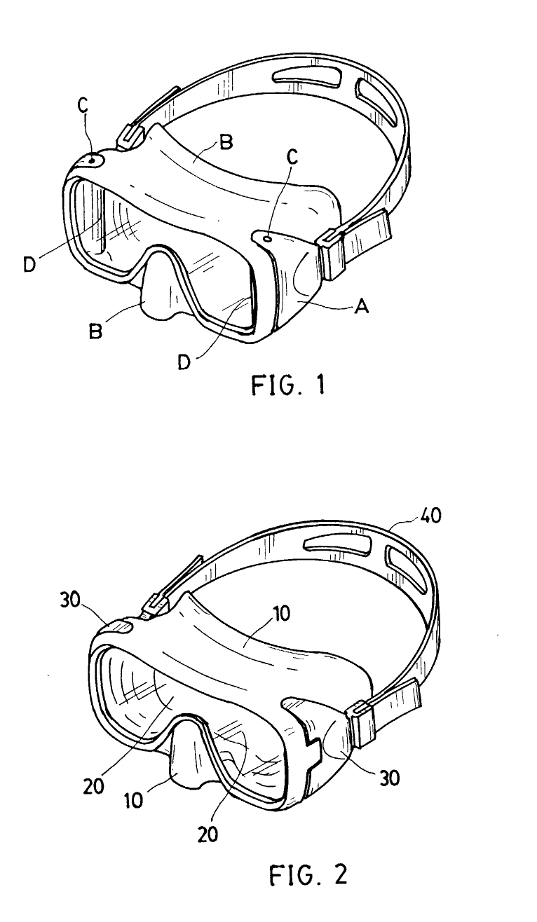

### DESCRIPTION OF THE PREFERRED EMBODIMENT

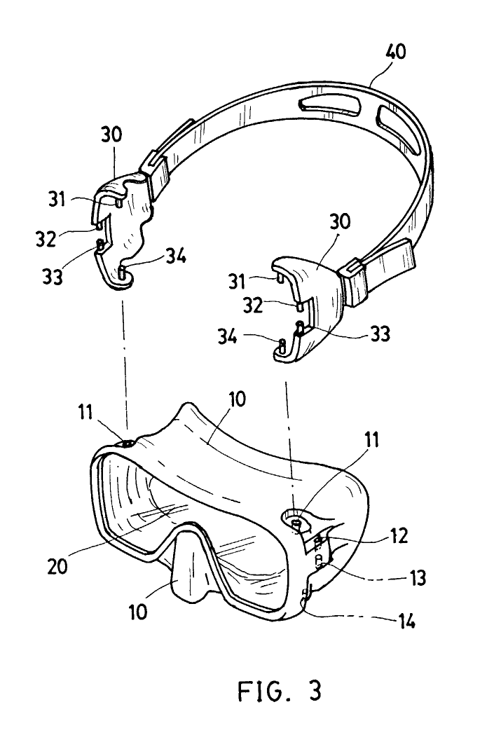

As shown on FIG. 2 and FIG. 3, the structure of the swimming mask of the present invention includes a mask body 10, lens 20, buckles 30 and a strap 40. Wherein said mask body 10 is made by soft material and lens 20 are made by tempered glasses or the like material. Said mask body 10 and at least a lens 20 can be integral during forming process. Said buckle 30 and strap 40 can be connected and the looseness can be adjusted for swimmer's wearing. The structure and combination of buckles 30 and strap 40 are various but this is not the subject matter of the present invention. The feature of the present invention is the combine structure of buckle 30 and mask body 10.

On the mask body 10 near the outside of lens 20, there are providing plurality of pin-holes 11, 12,13 and 14 which locating at the status of “the least misshape of mask body 10 when bearing the pulling strength from buckle 30”.

The structure of buckle 30 is made by hard material and the shape can be similar to conventional one. On the buckle30, provides plurality of pin 31, 32, 33 and 34. By the easily misshape of soft mask body 10, which enable said pins insert into said pin-holes 11, 12, 13 and 14 and fixed stably. When complete the insertion, the incoming strength of buckle 30 will be shared to each pin-hole 11, 12, 13 and 14 and the misshapen of mask body 10 can be reduced.

In conclusion, the design feature of multiple fasten points between the mask body 10 and buckle 30, which effectively spread and share the strength coming from buckle 30 and bore by mask body 10 and reduce the misshapen of mask body 10. The result and advantage is that the mask body is not easily misshaped caused by strength from operating and adjusting and further provides a wider view angle.

What is claimed is:

1. An improvement of buckle of swimming mask, said swimming mask comprising a soft mask body, at least a lens. Buckle and strap; said mask body and lens are combined integral; the buckle are provided to the mask body in larger area which enable the mask body not easy to misshape which the buckle bearing the strength.

2. The improvement of buckle of swimming mask as claimed in claim 1, said buckle having plurality of pins which inserting the corresponding pin-holes adopted on the mask body.
